(19) United States
(12) Patent Application Publication
(10) Pub. No.: US 2010/0136603 A1
(43) Pub. Date: Jun. 3, 2010

(54) KIT AND A METHOD FOR EVALUATING TOXICITY USING SPORE RELEASE BY THE GREEN ALGA ULVA

(75) Inventors: Tae-Jun Han, Incheon (KR); Young-Seok Han, Incheon (KR)

Correspondence Address: AMPACC Law Group, 3500 188th Street S.W., Suite 103, Lynnwood, WA, 98037, US

(73) Assignee: INDUSTRY-ACADEMIC COOPERATION FOUNDATION, University of Incheon, Incheon (KR)

(21) Appl. No.: 12/633,455

(22) Filed: Dec. 8, 2009

Related U.S. Application Data
(63) Continuation of application No. PCT/KR2008/006398, filed on Oct. 30, 2008.

(51) Int. Cl. C12Q 1/02

(52) U.S. Cl. 435/29

(57) ABSTRACT

The present invention relates to a kit and a method for evaluating toxicity of a waterbody sample by a spore release of a green alga Ulva. The kit for evaluating the toxicity of a waterbody sample comprises a capsule-type bioware that includes the leaf of the green alga Ulva prepared in a coin shape; a small cylinder for measuring a volume of a waterbody sample; an artificial salt for controlling growth conditions of the leaf of the green alga Ulva; a dividing measurement film or magnifying glass for measuring the area with changed color into white in the leaf of the green alga Ulva; and a standard toxic solution. The toxicity is evaluated based on the color change (particularly, the change into white color) due to the spore release of the green alga Ulva. Therefore, the toxicity of a waterbody sample can be easily and accurately evaluated by experts and unskilled personnel at low cost, rapidness and preciseness

### TECHNICAL FIELD

The present invention relates to a kit for evaluating toxicity by a spore release of a green alga Ulva, which comprises a capsule-type bioware that includes the leaf of the green alga Ulva prepared in a coin shape; a small cylinder for measuring a volume of a waterbody sample; an artificial salt for controlling growth conditions of the leaf of the green alga Ulva; a dividing measurement film or magnifying glass for measuring the area with changed color into white in the leaf of the green alga Ulva; and a standard toxic solution. In addition, the present invention relates to a method for evaluating toxicity by a spore release of a green alga Ulva, in which the toxicity is evaluated based on the color change (particularly, the change into white color) due to the spore release of the green alga Ulva.

### BACKGROUND ART

The rapid rise in industrialization has led to increases in toxicants in water bodies, which in turn causes novel problems. This gradual destruction of ecosystems due to environmental pollution (terrestrial and aquatic), is a matter of concern to all life forms including humans. Hence, regulations regarding environment protection and ecosystem restoration have been developed globally to help mitigate the effects of environment pollution, and particularly water pollution. However, the existing regulations need to be improvised regularly, i.e. these regulations should include latest toxicity testing methods which may either be more suitable, less time consuming, easy to perform or may aid in testing these new-fangled chemicals as well as the routine chemicals being tested. Thus, there is a need to develop new methods that can easily detect and quantify environmental pollution. Amongst the methods used, a bioassay is most frequently the preferred option as it involves exposing a living organism to harmful toxicants and measurement of a biological response such as death, growth, hatching, and fertilization thereafter. For example, when a living organism is exposed to a harmful material in an aqueous solution, it either dies or a growth pattern different from that of a “Comparative Example” or “control group” (experiment to which harmful material is not added) is obtained, which can help to determine the toxicity of the material tested.

The above method is a practical alternative for estimating toxicity, considering reasonable and functional aspects, as compared to toxicity estimation by measurements using different concentrations of each material and conducting known chemical analyses (which involves expensive equipment, chemicals, and skilled staff to undertake the analyses). Moreover, using chemical analysis, it is difficult to analyze toxicants, especially in cases where the inflow component of the toxic material is not known, and the component and the concentration change due to reaction in the sample, or in cases when the sample has moved far from the source. According to the proposed bioassay method, a lethal concentration (LC50), a half effective concentration (EC50) or similar units are calculated, and, based on these values, the relative quality of the toxicant and the toxicity of the material in the environment are determined and compared with each other.

The living organism that is used in a bioassay is selected according to the usefulness of the species, its sensitivity to toxic materials, the consistency of the reaction, resistance in respects to environmental factors, ecological importance, worldwide occurrence and availability, etc.

Methods using fish, invertebrate animals, or protozoa require professional personnel to conduct the analysis and to run expensive equipments. Additionally, the sensitivity of the test organism differs with the type of toxicant. Currently, rotifera, Cladocera, Pontogeneia rostrata, Photobacterium phosphoreum. etc. are amongst the most popular living organisms used in toxicity bioassays in most countries e.g. United States, countries in Europe. However, this method partially estimates toxicity of certain toxicants and there is always scope for the use of other specific living organisms to be used in bioassays, and practically all living things on the Earth could be used as tools for ecotoxicity measurements.

For a method to be registered in international ISO and OECD, it is necessary that it should be a significantly improved technology because of complexity of detection of toxicants, and it should overcome faults like instability of sample, low sensitivity, limit in area of use, difficulty in procurement, high cost, difficulty in continuous cultivation, supplying and management of the test organism. Bioassay methods qualitatively and quantitatively evaluate toxicants and overcome disadvantages and errors occurring in physico-chemical analysis.

Known kits for evaluating aquatic toxicities have several disadvantages including their high cost, complex measurement and operation procedures, and requirement of an expert to perform the test. In contrast, the green alga Ulva (particularly, Ulva pertusa) occurs worldwide, and is easy to cultivate and procure. The present inventors had formerly developed a pioneering method to estimate aquatic toxicities using a green alga Ulva (see Korean Patent Registration No. 653.100). The thallus of the green macroalga Ulva develops from gametes that live like plankton for a predetermined period and then attach to a predetermined substrate, and grow into a fully mature thallus. This former easy and economic process for estimation of toxicants flowing into seawater is developed based on the principle that the degree of toxicity of the seawater is inversely proportional to the ratio of formation and emission of gametes (reproduction ratio). Since this method had twice the sensitivity of the known toxicity estimation method using light emitting bacteria, rotifera, etc., which is commercialized in the USA or Europe, even lower concentrations of toxicants could be detected with high accuracy and sensitivity. Moreover, both the olive and white colors indicating reproductive phases of green alga Ulva were to be evaluated by the naked eye and image analysis in the previous method. Thus, shades of olive to white needed to be considered; hence precise estimation would vary from person to person, i.e. it would depend on the observer. But, in the present method, the same authors suggest the use of a single white color, which can be easily identified as an indication of reproduction. This would help in avoiding discrepancy in the results.

Taking into account this discrepancy, the present inventors conducted extensive studies to improve the testing technology. This novel and developed technology has been suggested in the present investigation. The modification involves evaluation of toxicity based on a vivid color change (white color) obtained when spores of a green alga Ulva are released. Further, this simplified invention includes and emphasizes the use of a kit and a specific method for evaluating toxicity.

### DISCLOSURE

### Technical Problem

the present invention aims to provide a kit for evaluating toxicity of water by a spore release of a green alga Ulva, which is characterized by its simplicity, effortlessness, excellent sensitivity, precision and inexpensiveness; it can be easily used by experts and unskilled personnel alike.

In addition, the present invention aims to provide a method for evaluating toxicity of water by a spore release of a green alga Ulva, which is characterized in that a measuring endpoint is apparent; it can be easily used by experts and unskilled people

### TECHNICAL SOLUTION

The present invention provides a kit for evaluating toxicity of water by a spore release of a green alga Ulva, which comprises a capsule-type bioware that includes the leaf of the green alga Ulva prepared in a coin shape; a small cylinder for measuring a volume of a waterbody sample; an artificial salt for controlling growth conditions of the leaf of the green alga Ulva; a dividing measurement film or magnifying glass for measuring the area with changed color in the leaf of the green alga Ulva; and a standard toxic solution.

The kit includes plastic forceps and gloves which aid in performing the toxicity evaluation, a reference diagram to note the change in color of the leaf (thallus), a CD demonstrating the procedure to conduct the test using the kit, and a CD regarding statistical analysis for experts.

Generally, the reproduction of green alga Ulva occurs rapidly in a non-polluted environment and the color of the thallus that is called a leaf changes from an initial light green color to dark olive color, and then finally to white color. However, the reproduction ability of this alga is reduced in polluted water or in the presence of toxicants, which in turn causes a reduction in the degree of color change. Based on this principle, the leaves of the green alga Ulva prepared in a coin shape are exposed to the toxicant to be tested followed by direct observation by the naked eye or image capturing using a camera. More particularly, the area of the leaf showing change into color (i.e. white color) is measured using the measuring film and magnifying glass provided in the kit for visible analysis or using a camera for image analysis and is compared to the area of the white thallus that is shown in the control group not containing the toxicity source; thereafter the degree of toxicity of the sample is determined.

The principal steps involved in the present invention can be described as follows: preparation of the 24-well flat culture plate→dispensing a waterbody sample having toxicants, using the half dilution method (100%, 50%, 25%, 12.5%, and 6.25%)→placing a coin shaped, green alga Ulva leaf into each well→incubation and cultivation of the alga in 24-well flat culture plate containing the toxic material for 96 hours→calculation of the area of the leaf that has changed to a white color→comparison with that of control leaf i.e. not exposed to toxicants→analysis and calculation using a statistical program→predicting the degree of toxicity of the sample tested. A coin shaped, green alga Ulva leaf is placed in the 24-well flat culture plate containing a waterbody sample having toxicants and it is cultivated under the conditions of the photon irradiance in the range of 30 to 200 μmol photon/m²·s, the pH in the range of 7 to 9, the salinity in the range of 25 to 35% and the temperature in the range of 15 to 20° C. After a 96-h culture, spore release is quantified based on the total area of the leaf and area of the leaf which has changed to a white color. This can either be done visually using the naked eye with the help of a measurement film and magnifying glass, or by capturing and storing its image using a camera.

The toxicity is calculated indirectly by the following Equation:

[1−(A−B)/A]×100(%)

A: area of the leaf changed into white color in the control group (no toxicant)

B: area of the leaf changed into white color in the experimental group (exposed to the toxicant in solution form).

The above Equation represents the percentage of the area of the leaf changed into white color in the experimental group relative to the area of the leaf changed into white color in the control group, and the toxicity is calculated by subtracting the percentage of The above Equation from 100%.

The present invention provides a method for evaluating toxicity of water by the spore release of the green alga Ulva, which comprises the following steps of (a) adding the leaf of the green alga Ulva that is divided into 16 parts and has a coin shape to a 24-well flat culture plate that includes a waterbody sample containing toxicants; (b) culturing it for 96 hours in the 24-well flat culture plate under the conditions of photon irradiance in the range of 30 to 200 μmol photon/m²·s, pH in the range of 7 to 9, salinity in the range of 25 to 35% and temperature in the range of 15 to 20° C.; (c) when the spore release is performed, measuring the area of the leaf where the color has changed into white based on the total area of the leaf by using a dividing measurement film or magnifying glass; and (d) determining the toxicity by calculating and analyzing data.

Determination of the toxicity of the sample solution can be made by calculating and analyzing data using the software provided.

Toxicities of waterbody samples selected from sea water, river water, lake water, wastewater, discharged water, dirty water, sludge elution water, soil elution water, or loess elution water can be evaluated using spore release by green alga Ulva. The types of toxicant that can be evaluated using this method include 12 metals such as silver (Ag), arsenic (As), copper (Cu), cadmium (Cd), cobalt (Co), chromium (Cr+6), iron (Fe), mercury (Hg), manganese (Mn), nickel (Ni), lead (Pb), and zinc (Zn); volatile organic compounds (VOC); polycyclic aromatic hydrocarbons (PAH); polychlorobiphenyls (PCB); and TBTO [bis(tri-n-butyltin) oxide].

The standard toxic solution, preferably a copper solution (CAS No. 7440-50-8. Cu standard solution), is used to check the state of the leaf of the green alga Ulva. If the state of the leaf of the green alga Ulva is verified by means of the standard solution, the reliability of the results of the experiment will be guaranteed.

The prerequisite conditions of cultivation of the coin shaped green alga Ulva leaf (thallus) include a period of 96 hours in a 24-well flat plate culture medium under the condition of photon irradiance in the range of 30 to 200 μmol photon/m2·s, pH in the range of 7 to 9, salinity in the range of 25 to 35% and temperature in the range of 15 to 20° C.

In the previous method a 6-well flat culture plate was used and approximately 10 ml of a test solution was required; however, in the present method using the spore release of the green alga Ulva, a 24-well flat culture plate medium and 2.5 ml test solution is required, which increases the possible of number of repetition and hence enhances the precision, improves objectivity and reduces the amount of the sample solution whose toxicity is to be evaluated.

In addition, in the previously defined method using the green alga Ulva, the reproduction ratio is estimated visibly by arbitrarily dividing the coin shaped thallus into 4 imaginary equal parts. In the present invention, however, a measurement film with 16 circular girds and magnifying glass are employed to estimate the spore release of the green alga Ulva, enhancing the accuracy of the test and increasing the possibility of obtaining precise results. Furthermore, the period for testing has been shortened to 4 days in this invention (which saves about 24 hours) as compared the previous invention where 5 days were required.

### ADVANTAGEOUS EFFECTS

According to the present invention, a small volume of sample solution (approximately 2.5 ml) is required along with a 24-well flat culture plate, which increases the possible number of repetitions in the test, thus increasing the sensitivity of the test and largely reducing the volume of the estimation solution that is added per each experiment. Besides, the accuracy of the measured value is increased by dividing the thallus of the green alga Ulva into 16 parts, and the toxicity evaluation period has been reduced by about 24 hours as compared to the method using the reproduction ratio of the green alga Ulva.

Therefore, the toxicity of water can be easily and accurately evaluated by experts and unskilled personnel at low cost, rapidness and preciseness

### DESCRIPTION OF DRAWINGS

FIG. 1 is a flow chart that illustrates a method for evaluating the toxicity of a waterbody sample by a spore release of a green alga Ulva as one embodiment of the present invention:

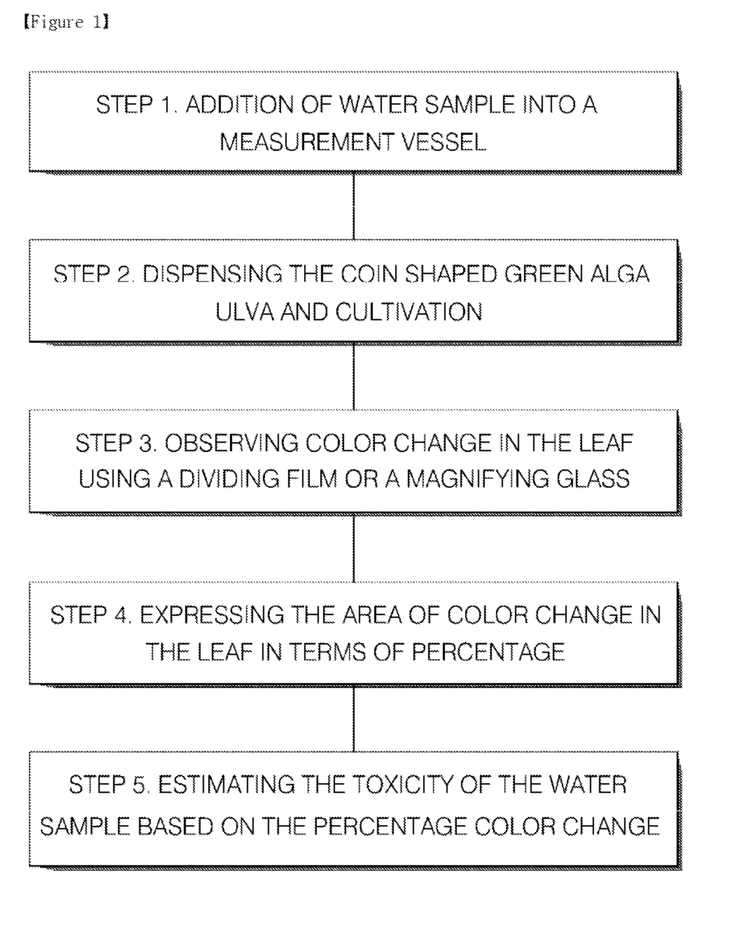

FIG. 2 illustrates a test principle of a kit for evaluating the toxicity of a waterbody sample by a spore release of a green alga Ulva as one embodiment of the present invention:

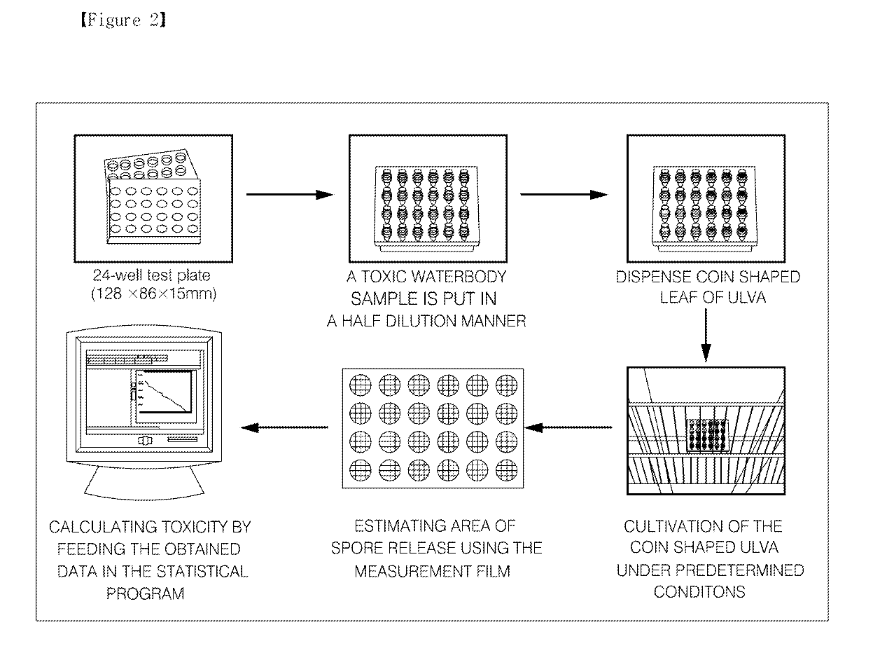

FIG. 3 is a pictorial configuration of a kit for evaluating the toxicity of a waterbody sample by a spore release of a green alga Ulva as one embodiment of the present invention; and

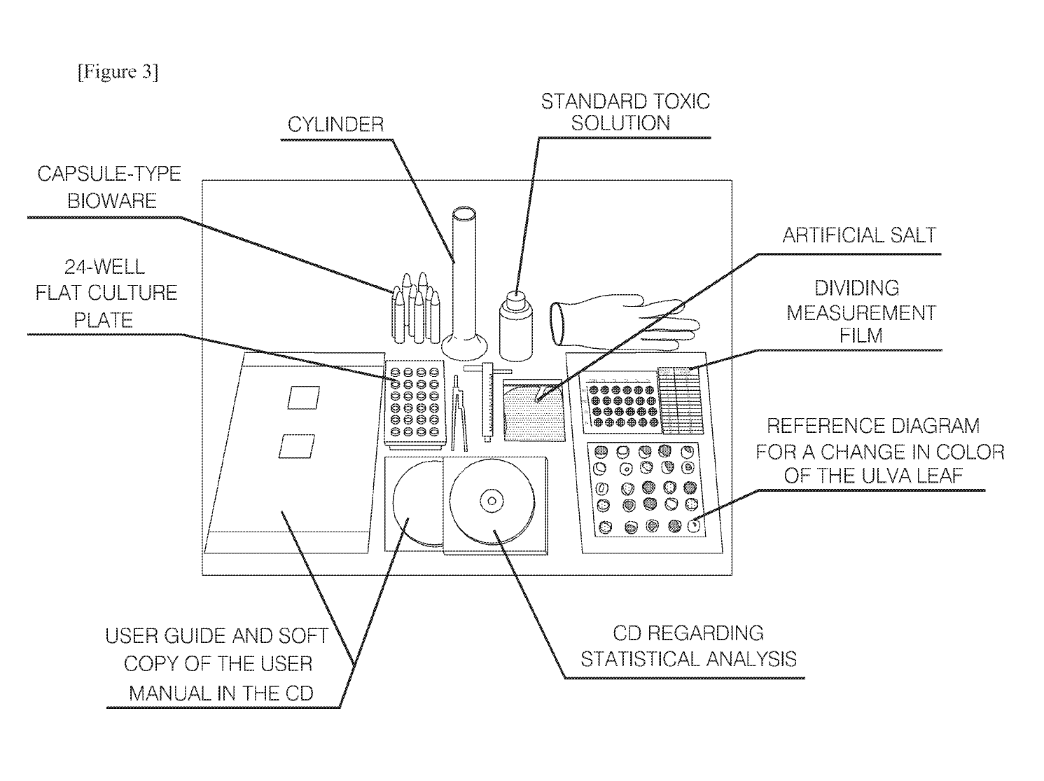

FIG. 4 illustrates an area calculation table of the results obtained by a dividing measurement film or a magnifying glass included in the kit, which is used to estimate the toxicity of a waterbody sample.

### BEST MODE

The coin-shaped green alga Ulva leaf was placed into the 24-well flat culture plate that contained medium mixed with a water sample contaminated with the standard toxic solution (copper solution). In this case, the standard toxic solution (copper solution) was dispensed in a half dilution manner (100%, 50%, 25%, 12.5%, and 6.25%). The green alga Ulva leaf was then cultivated in the 24-well flat plate culture medium for 96 hours under the conditions of photon irradiance of 100 μmol photon/m2·s, pH of 8.0, salinity of 35% and temperature of 15° C. Subsequently, the area of the leaf that had changed into white color was measured and compared to total area of the leaf using a 16-dividing area measurement film. The toxicity was then estimated by calculating and analyzing the data using a program.

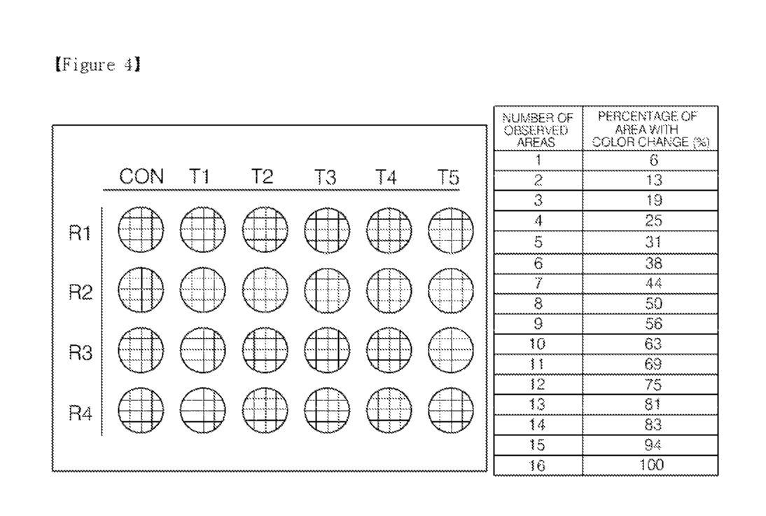

As shown in FIG. 4, T1 to T5 comprises the subject sample, and R1 to R5 indicates the number of repetitions in the test. FIG. 4 elucidates visible division of the coin shaped green alga Ulva into 16 parts using a measurement film, wherein each area represents a percentage, and a ratio of the 16 areas was observed, thereby easily estimating the toxicity. For example, if 9 of the 16 areas changed into white color, it means that the percentage of the area with white color by a spore release was 56%, which in turn means that the degree of toxicity 44%.

### MODE FOR INVENTION

The present inventors collected Ulva pertusa Kjellman from the coast of Ahnin in Korea, and the leaf (thallus) of Ulva pertusa Kjellman was cut to have a coin shape having a diameter of 6 mm, and dispensed in the plastic cell plate containing 10 ml artificial sea water (35%) medium (Coralife, Energy Savers, California. USA) supplemented with 1 mM KNO₃ and 0.1 mM K₂HPO₄. Ulva pertusa Kjellman leaf was cultivated Under the optimum environmental condition (100 μmol photon/m²s, 12:12h L:D photoperiodism, 15° C., 35%), determined after testing under the following conditions: photon irradiance in the range of 5 to 200 μmol photon/m²·s, pH in the range of 4 to 9, salinity in the range of 5 to 55%, and temperature in the range of 5 to 25° C.

The toxicity test was then carried out by adding toxic materials (toxicants) into the medium, keeping the other environmental conditions constant. The used toxic materials and the target concentration are described in the following Table 1.

TABLE 1

| toxic material | target concentration | |
|---|---|---|
| Cd | 0.0375-0.6 | mg/L |
| Cu | 0.0125-0.2 | mg/L |
| Pb | 0.125-2 | mg/L |
| Zn | 0.125-2 | mg/L |
| acetone | 5-80 | mg/L |
| ethanol | 5-80 | mg/L |
| formaline | 0.438-7 | μl/L |
| methanol | 5-80 | ml/L |
| TBTO | 0.0125-0.2 | mg/L |

After cultivated for 96 hours, the thallus having the coin shape was collected, and the area with white color was measured by using the microscope (Axioscope) having a magnification of 400× and the image analysis system (MV200, Samsung, Korea).

The toxicity of heavy metals was tested using standard mother liquid of cadmium, copper, lead and zinc prepared in deionized water that was acidified with 1N HCl or 1M HNO₃. The mother liquid of TBTO was prepared in acetone that was mixed with the sea water (0.01%); here an acetone control group in the same concentration was also tested. The toxicities of volatile organic compounds (VOC) [acetone, ethanol, formalin and methanol) were tested by adding aqueous volatile organic compounds (VOC) to the medium to achieve the target concentration.

The non-effective concentration (NOEC; the maximum concentration having no difference in respects to the control group), which was estimated by linear interpolation method, had 95% confidence interval and the half effective concentration (EC₅₀) were also calculated.

The toxicity of the heavy metal, which was estimated by EC₅₀, was in the order of Cu>Cd>Zn>Pb. Meanwhile, the toxicity of the volatile organic compound (VOC) was estimated in the order of formaline>methanol>ethanol>acetone. The half effective concentration (EC₅₀) of formaline having the strongest toxicity was 0.788 μl/L.

### INDUSTRIAL APPLICABILITY

According to the kit and the method for evaluating toxicity in water using spore release of a green alga Ulva, the following benefits may be derived in terms of technical, environmental, industrial and economic fields.

### Technical Field

Detection of harmful materials or toxicants and improvement of water quality, which in turn would aid in preserving and maintaining ecological environment and public water bodies.

Preparation of a countermove through prediction of toxicity of harmful material and hence preventing future changes in environment.

Improvement of environmental standards by avoiding use of known physicochemical methods, which may cause secondary pollution.

Reduction of manpower and cost that are usually involved to conduct laborious physico-chemical analysis to detect individual harmful material.

Evasion of economic loss resulting from importing samples and technologies, and demolishing evils of backwardness in technology, improving the image of the country by providing a domestic practical technology which wipes out the image of lack of research conducted and lack of investment in domestic techniques.

Realization of commercialization of a biological toxicity estimation methods having international competitiveness.

Realization of a user-specific biological toxicity estimation system.

Inspiration for development of other similar eco-friendly toxicity assays.

### Environmental Field

Contribution to preserve the ecosystem, human health and welfare.

Prevention of secondary pollution by an environmentally friendly toxicity evaluation method.

Realization of an eco-friendly, novel environment management system.

Use of an abundantly available natural resource.

### Economical and Industrial Fields

Protection of human health and preservation of ecosystem through environmental management using a cost effective health sanitation and environment preservation technique.

Reduction of foreign investment and development of domestic technology which would generate income and could be used locally with ease.

Evasion of dependence on skilled manpower or experienced personnel usually involved in toxicity assays.

Activation of related technology fields and induction of industrial groups.

Ensuring of export competitiveness by manufacturing environmentally friendly products through advanced environment pollution management.

1. A kit for evaluating toxicity of water by a spore release of a green alga Ulva, the kit comprising:
a capsule-type bioware that includes the leaf of the green alga Ulva prepared in a coin shape;
a small cylinder for measuring a volume of a waterbody sample:
an artificial salt for controlling growth conditions of the leaf of the green alga Ulva;
a dividing measurement film or magnifying glass for measuring the area with changed color in the leaf of the green alga Ulva; and
a standard toxic solution.

2. The kit according to claim 1, wherein the leaf of the green alga ulva is divided into 16 parts.

3. The kit according to claim 1, wherein in respects to the growth conditions of the leaf of the green alga Ulva, photon irradiance is in the range of 30 to 200 μmol photon/m²·s, pH is in the range of 7 to 9, salinity is in the range of 25 to 35%, and temperature is in the range of 15 to 20° C.

4. The kit according to claim 1, wherein the standard toxic solution is a copper solution.

5. A method for evaluating toxicity of water by a spore release of a green alga Ulva, the method comprising the following steps of:
(a) adding the leaf of the green alga Ulva that is divided into 16 parts and has a coin shape to a 24-well flat culture plate that includes a waterbody sample containing toxicants;
(b) culturing it for 96 hours in the 24-well flat culture plate under the conditions of photon irradiance in the range of 30 to 200 μmol photon/m²·s, pH in the range of 7 to 9, salinity in the range of 25 to 35% and temperature in the range of 15 to 20° C.;
(c) when the spore release is performed, measuring the area of the leaf where the color has changed into white based on the total area of the leaf by using a dividing measurement film or magnifying glass; and
(d) determining the toxicity by calculating and analyzing data.

6. The method according to claim 5, wherein the waterbody sample is sea water, river water, lake water, wastewater, discharged water, dirty water, sludge elution water, soil elution water, or loess elution water.

7. The method according to claim 5, wherein the toxicants are selected from heavy metals including copper (Cu), cadmium (Cd), lead (Pb), and zinc (Zn); volatile organic compounds (VOC); multicycle aromatic hydrocarbons (PAH); polychlorobiphenyls (PCB); and TBTO [bis(tri-n-butyltin) oxide].

8. The method according to claim 5, wherein the waterbody sample having toxicants is put in a half dilution method (100%, 50%, 25%, 12.5%, and 6.25%).

9. The method according to claim 5, wherein the standard toxic solution containing copper is used to check the state of the leaf of the green alga Ulva.
